(19) United States
(12) Patent Application Publication
(10) Pub. No.: US 2015/0065804 A1
(43) Pub. Date: Mar. 5, 2015

(54) CANNULA - OBTURATOR STRUCTURE

(71) Applicant: Covidien LP, Mansfield, MA (US)

(72) Inventor: Gennady Kleyman, Brooklyn, NY (US)

(21) Appl. No.: 14/446,890

(22) Filed: Jul. 30, 2014

Related U.S. Application Data
(60) Provisional application No. 61/871,020, filed on Aug. 28, 2013.

(51) Int. Cl. A61B 17/34; A61B 17/02

(52) U.S. Cl. CPC A61B 17/3423; A61B 17/0218; A61B 2017/3445
USPC 600/204

(57) ABSTRACT

A cannula includes a body portion having circumferential openings at the distal end thereof and a removable tip incorporated at the distal end of the body portion, the removable tip having circumferential tabs for releasably interlocking with the circumferential openings of the cannula. The removable tip serves as an obturator, and it is positioned within the distal end of the cannula, such that the cannula and obturator are combined into a single unitary cannula assembly.

### CROSS-REFERENCE TO RELATED APPLICATIONS

This application claims the benefit of and priority to U.S. Provisional Patent Application No. 61/871,020, filed Aug. 28, 2013, the entire disclosure of which is incorporated by reference herein.

### BACKGROUND

1. Technical Field

The present disclosure relates to surgical instruments. More particularly, the present disclosure relates to a cannula including an obturator structure incorporated therein in a pre-assembled manner.

2. Background of the Related Art

Access systems have been of particular advantage in facilitating less invasive surgery across a body wall and within a body cavity. This is particularly true in the case of abdominal surgery where access systems have provided working channels across the abdominal wall to facilitate the use of instruments within the abdominal cavity.

The access systems of the past typically include a cannula, which defines the working channel, and an obturator which is used to place the cannula across the abdominal wall. The obturator is inserted into the working channel of the cannula and then pushed through the abdominal wall with a penetration force of sufficient magnitude to result in penetration of the abdominal wall. Once the cannula is in place, the obturator can be removed.

In the past, obturators have been developed with an intent to provide a reduction in the force required for penetration. Sharp blades have typically been used to enable the obturator to cut its way through the abdominal wall.

In some cases, shields have been provided with the obturators in order to sense penetration of the abdominal wall and immediately shield the sharp blades.

### SUMMARY

The following presents a simplified summary of the claimed subject matter in order to provide a basic understanding of some aspects of the claimed subject matter. This summary is not an extensive overview of the claimed subject matter. It is intended to neither identify key or critical elements of the claimed subject matter nor delineate the scope of the claimed subject matter. Its sole purpose is to present some concepts of the claimed subject matter in a simplified form as a prelude to the more detailed description that is presented later.

A cannula assembly in accordance with an embodiment of the present disclosure includes a cannula having a plurality of circumferential openings at a distal end thereof and an obturator having a proximal end and a distal end, and a plurality of circumferential tabs that lockingly engage the plurality of circumferential openings of the cannula.

In an exemplary embodiment, the distal end of the obturator includes a tip and the proximal end of the obturator includes a plurality of leg structures. Each of the plurality of leg structures of the obturator is configured to interlock with at least one latching mechanism of a retractor for disengaging the plurality of circumferential tabs of the obturator from the plurality of circumferential openings of the cannula.

In another exemplary embodiment, each of the plurality of leg structures of the obturator includes a snap profile for engaging the at least one latching mechanism of a retractor.

In yet another exemplary embodiment, the obturator is pre-assembled to interlock with the cannula.

In an exemplary embodiment, the obturator, in its entirety, is positioned at the distal end of the cannula, such that the obturator and cannula move together as one unit.

In another exemplary embodiment, the cannula has a first length and the obturator has a second length, the second length being less than half of the first length.

An obturator in accordance with an embodiment of the present disclosure includes a tip at a distal end thereof, a plurality of leg extensions at a proximal end thereof and a plurality of circumferential tabs positioned between the proximal and distal ends of the obturator.

In an exemplary embodiment, the plurality of circumferential tabs is adapted and dimensioned to engage a plurality of circumferential openings of a distal end of a cannula.

In another exemplary embodiment, the plurality of leg extensions is adapted and dimensioned to engage a plurality of collet fingers of a retractor for removing the obturator from a cannula. Each of the plurality of leg extensions includes a snap profile for engaging the plurality of collet fingers of a retractor.

In yet another exemplary embodiment, the obturator is configured to be pre-assembled, in a releasable manner, into an inner portion of a cannula.

A cannula in accordance with an embodiment of the present disclosure includes a head portion, a body portion having a proximal end and a distal end, a plurality of circumferential openings at the distal end of the body portion and a removable tip incorporated at the distal end of the body portion, the removable tip having a plurality of circumferential tabs for releasably interlocking with the plurality of circumferential openings.

In an exemplary embodiment, the removable tip is pre-assembled to interlock within an inner portion at the distal end of the body portion.

A method in accordance with an embodiment of the present disclosure includes the step of inserting a cannula assembly through a body wall of a patient, the cannula assembly including a cannula having a proximal end and a distal end and a plurality of circumferential openings at a distal end thereof and an obturator having a proximal end and a distal end, a plurality of circumferential tabs that engage the plurality of circumferential openings of the cannula and a plurality of leg extensions at a proximal end thereof, wherein the cannula has a first length and the obturator has a second length, the second length being less than half of the first length. The method also includes the steps of removing the obturator from the cannula by inserting a retractor into the proximal end of the cannula, the retractor including at least one latching mechanism that interlocks with the plurality of leg structures of the obturator and disengages the plurality of circumferential tabs of the obturator from the plurality of circumferential openings of the cannula and using the cannula as an access port for passage of surgical instruments through a body wall of a patient.

Further scope of applicability of the present disclosure will become apparent from the detailed description given hereinafter. However, it should be understood that the detailed description and specific examples, while indicating specific embodiments of the present disclosure, are given by way of illustration only, since various changes and modifications within the spirit and scope of the present disclosure will become apparent to those skilled in the art from this detailed description.

### BRIEF DESCRIPTION OF THE DRAWINGS

The above and other aspects, features, and advantages of the present disclosure will become more apparent in light of the following detailed description when taken in conjunction with the accompanying drawings in which:

FIGS. 1A-1C are perspective views of a removable obturator, having a plurality of circumferential tabs thereon, according to an embodiment of the present disclosure;

FIGS. 2A-2C are perspective views of a cannula illustrating a plurality of openings at a distal end thereof, according to an embodiment of the present disclosure;

FIGS. 3A-3C are perspective views of pre-assembling the obturator of FIGS. 1A-1C to the cannula of FIGS. 2A-2C, according to an embodiment of the present disclosure;

FIGS. 4A-4C are perspective views of a retractor, according to an embodiment of the present disclosure;

FIG. 4D is a cross-sectional view of the retractor of FIGS. 4A-4C, according to an embodiment of the present disclosure;

FIGS. 5A-5D are cross-sectional views of the retractor entering the cannula, connecting to the obturator, and removing the obturator from the cannula, according to an embodiment of the present disclosure;

FIGS. 5E-5H are perspective views of the retractor entering the cannula, connecting to the obturator, and removing the obturator from the cannula, according to an embodiment of the present disclosure; and

FIGS. 6A-6C illustrate different obturator tips, in accordance with various embodiments of the present disclosure.

The figures depict specific embodiments of the present disclosure for purposes of illustration only. One skilled in the art will readily recognize from the following discussion that alternative embodiments of the structures and methods illustrated herein may be employed without departing from the principles of the present disclosure described herein.

### DETAILED DESCRIPTION

Particular embodiments of the present disclosure are described hereinbelow with reference to the accompanying drawings; however, it is to be understood that the disclosed embodiments are merely exemplary of the disclosure and may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

Like reference numerals may refer to similar or identical elements throughout the description of the figures. As shown in the drawings and described throughout the following description, as is traditional when referring to relative positioning on a surgical instrument, the term “proximal” refers to the end of the apparatus which is closer to the user and the term “distal” refers to the end of the apparatus which is farther away from the user. The term “clinician” refers to any medical professional (i.e., doctor, surgeon, nurse, or the like) performing a medical procedure involving the use of embodiments described herein.

The word “exemplary” is used herein to mean “serving as an example, instance, or illustration.” Any embodiment described herein as “exemplary” is not necessarily to be construed as preferred or advantageous over other embodiments. The word “example” may be used interchangeably with the term “exemplary.”

Reference will now be made in detail to embodiments of the present disclosure. While certain embodiments of the present disclosure will be described, it will be understood that it is not intended to limit the embodiments of the present disclosure to those described embodiments. To the contrary, reference to embodiments of the present disclosure is intended to cover alternatives, modifications, and equivalents as may be included within the spirit and scope of the embodiments of the present disclosure as defined by the appended claims.

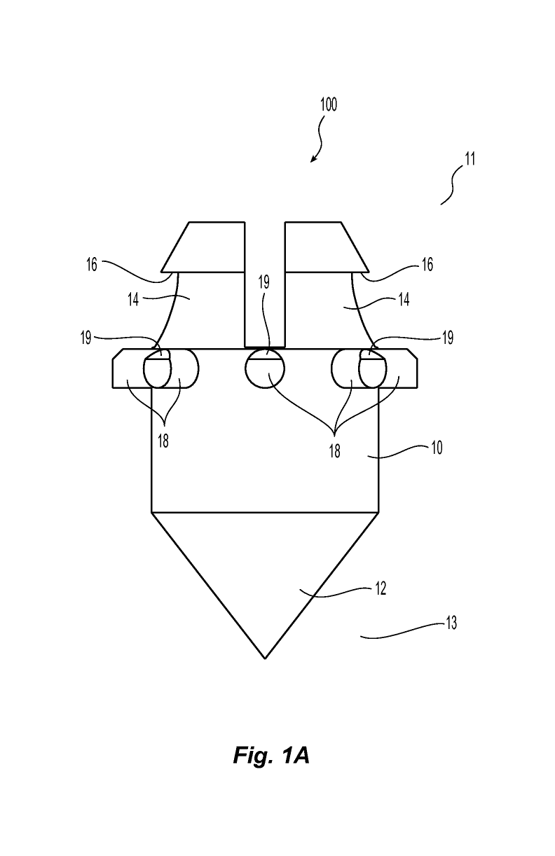

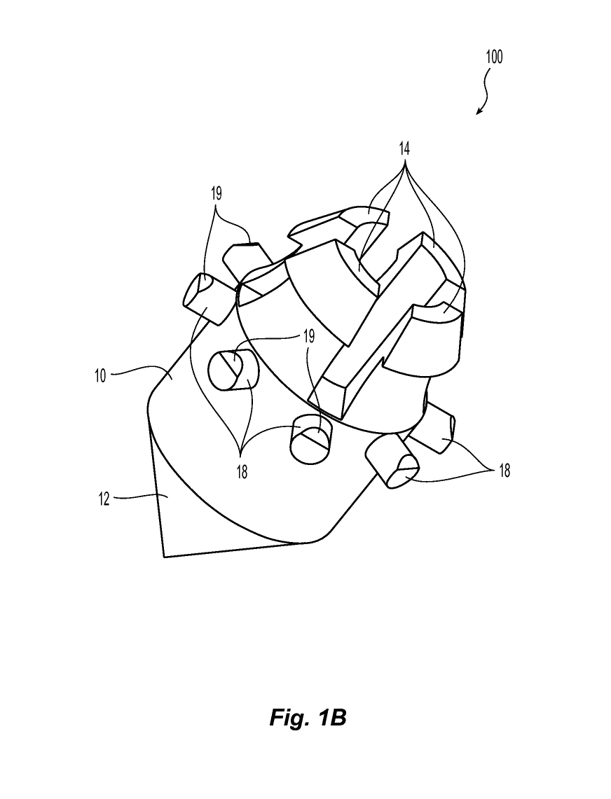

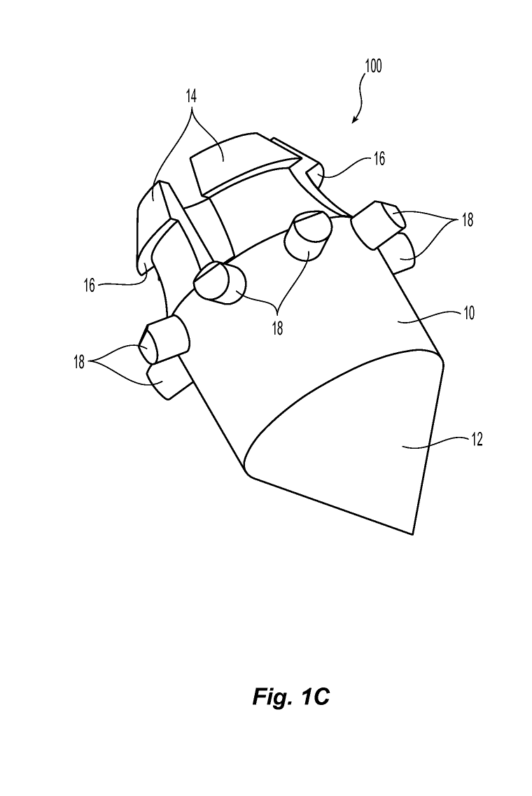

FIGS. 1A-1C present various views of a removable obturator 100, having a plurality of circumferential tabs 18 thereon, according to an embodiment of the present disclosure.

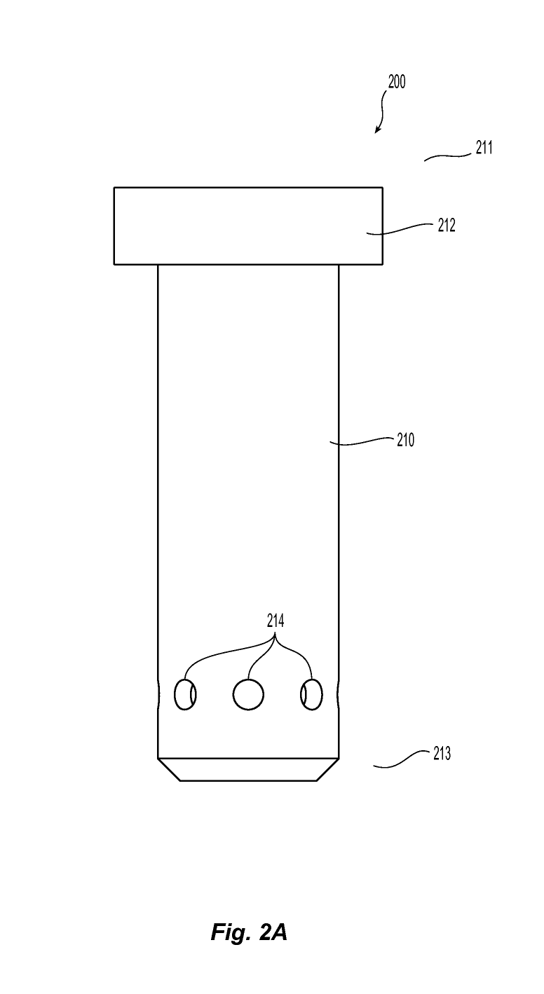

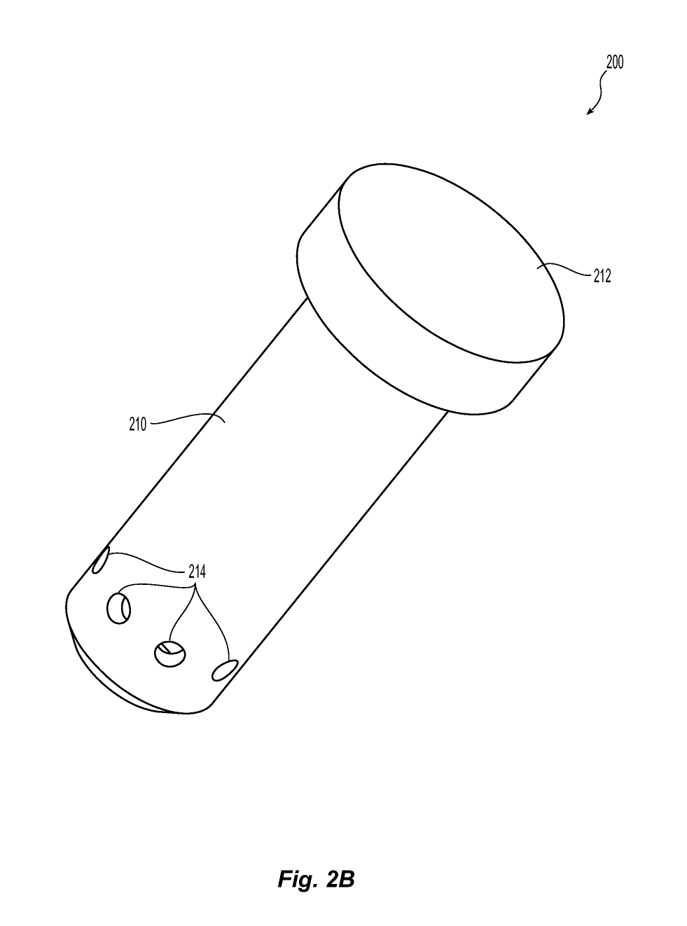

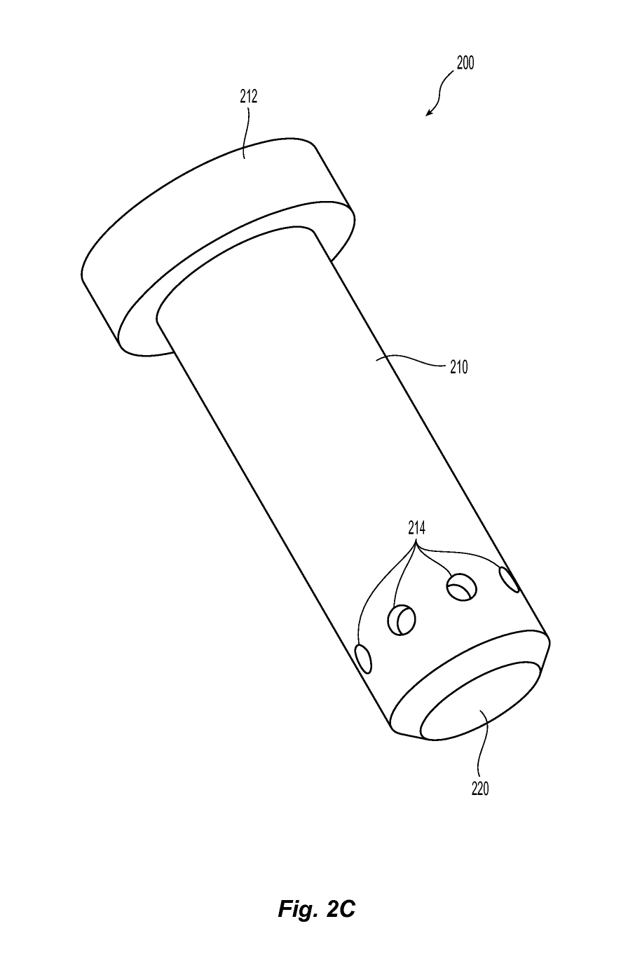

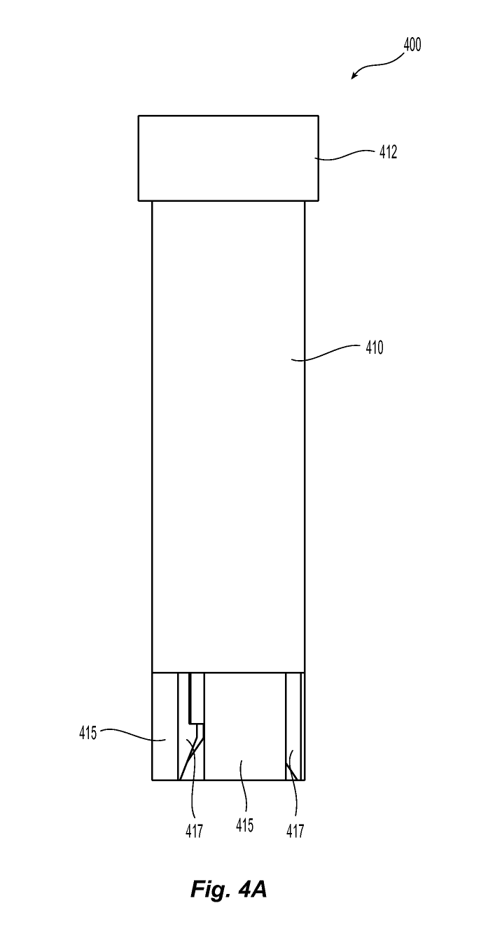

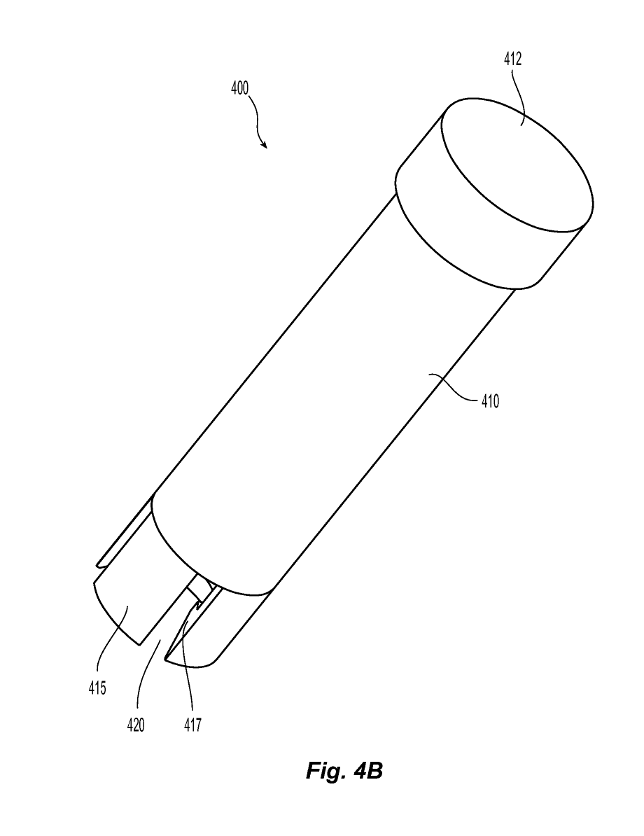

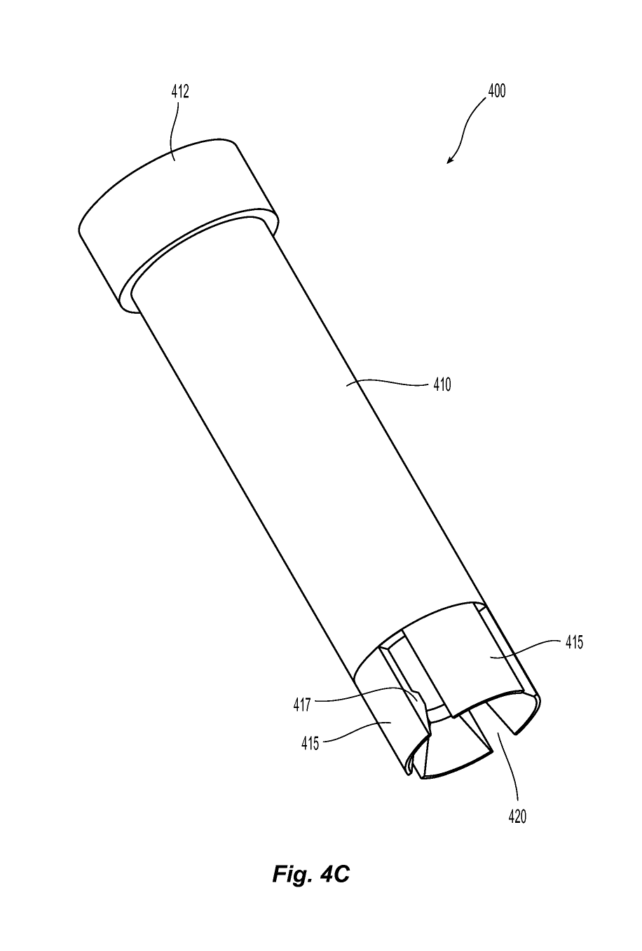

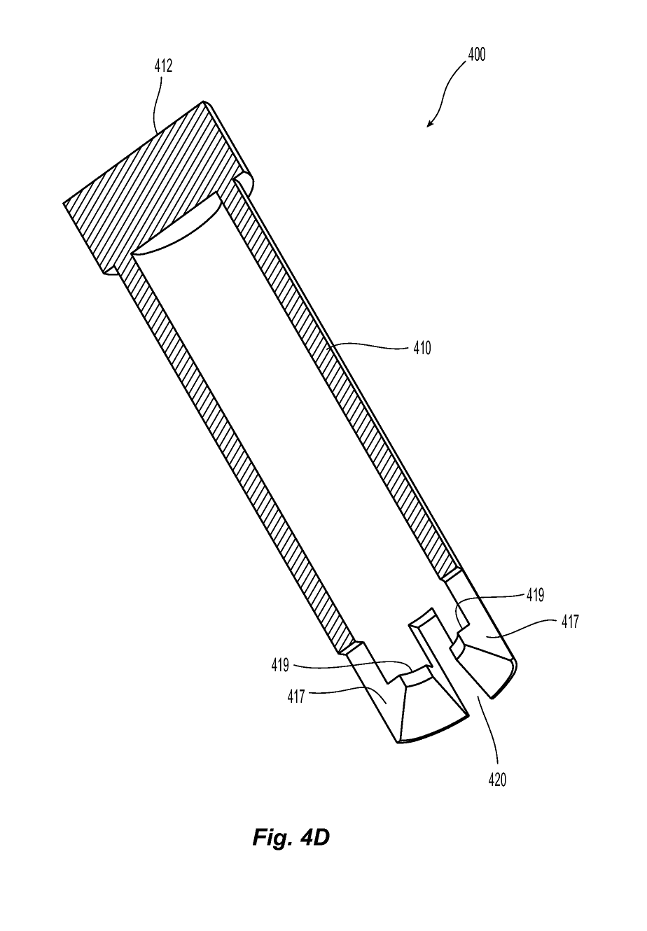

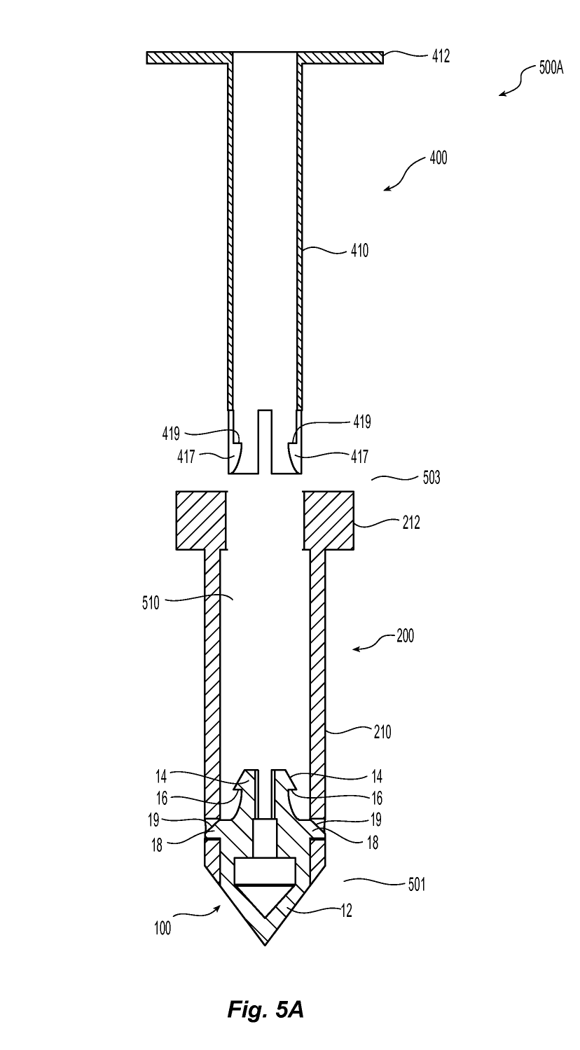

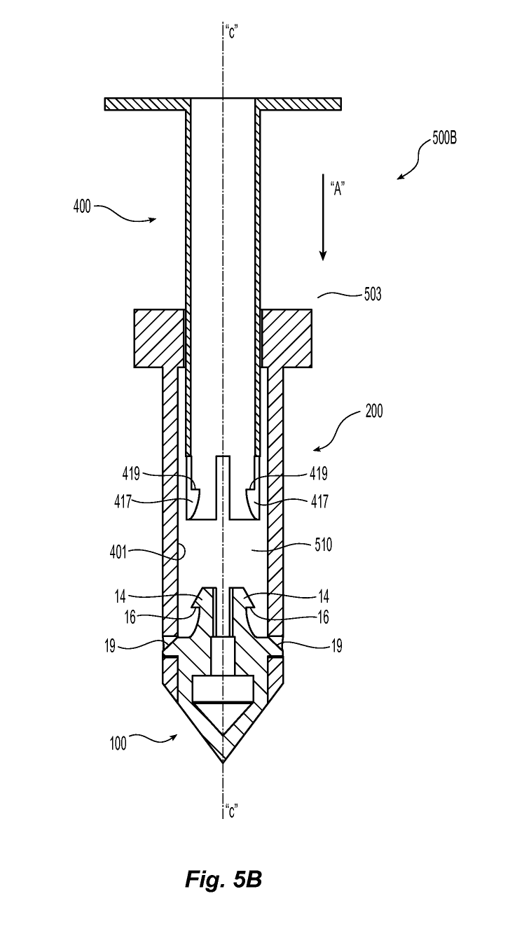

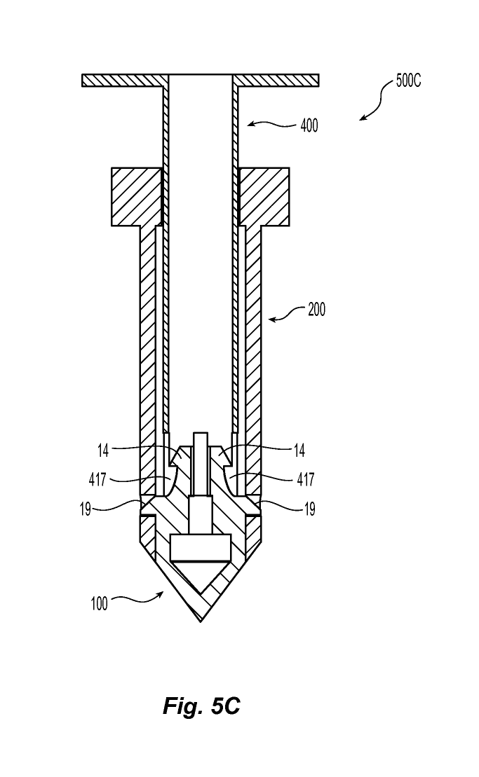

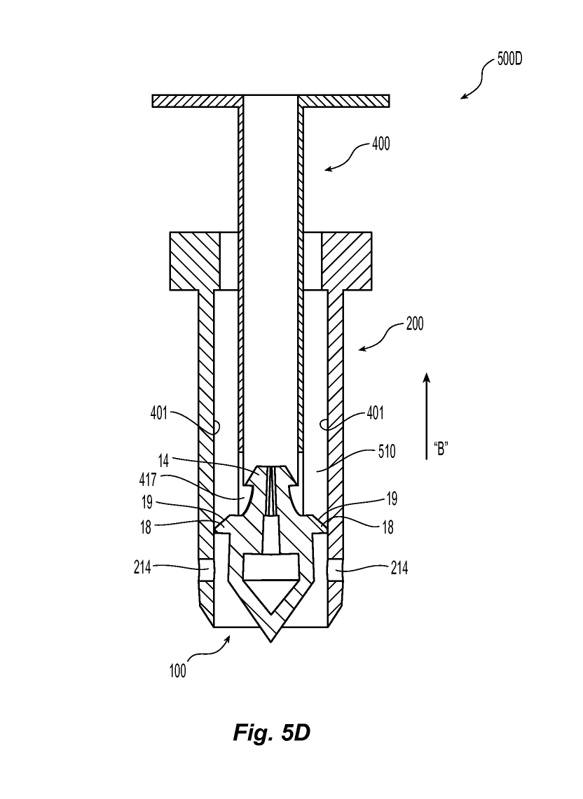

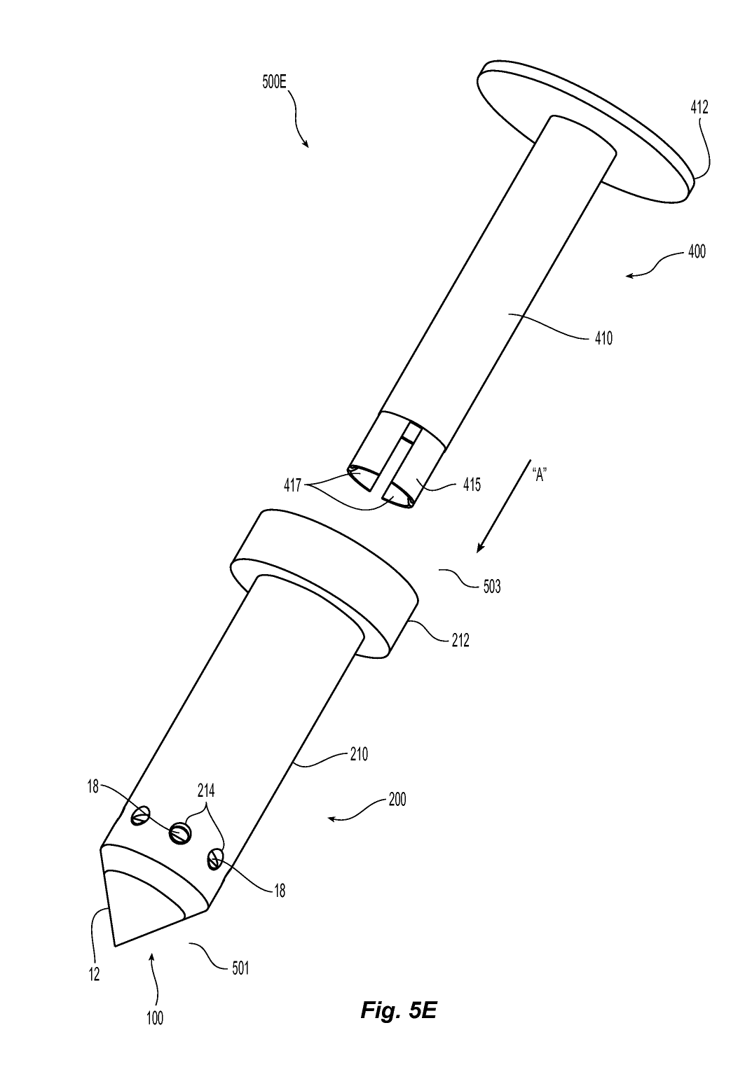

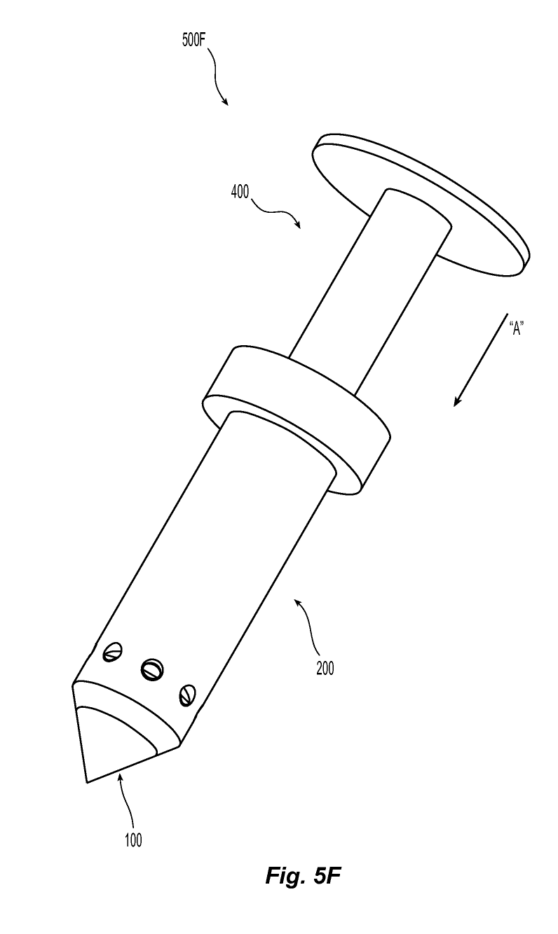

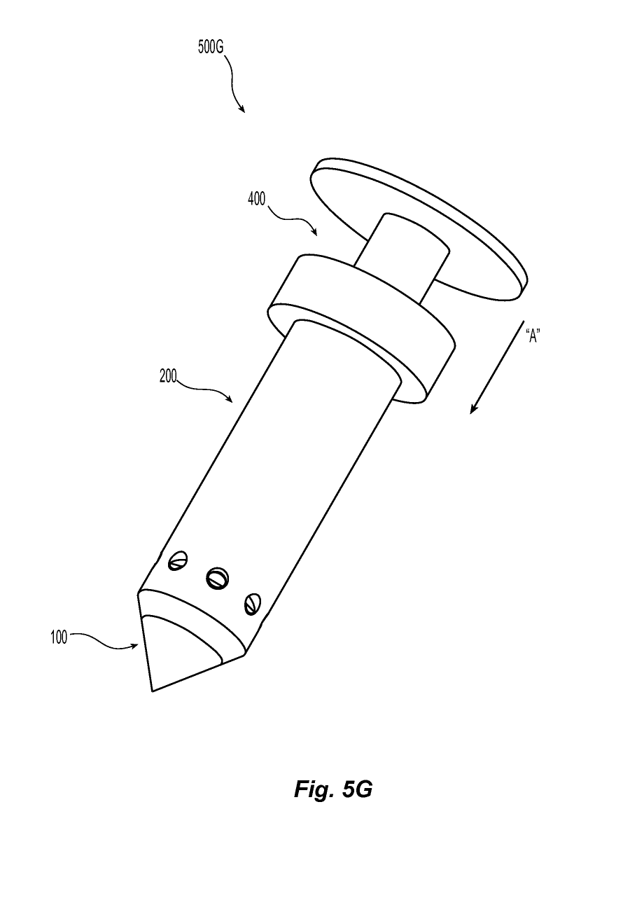

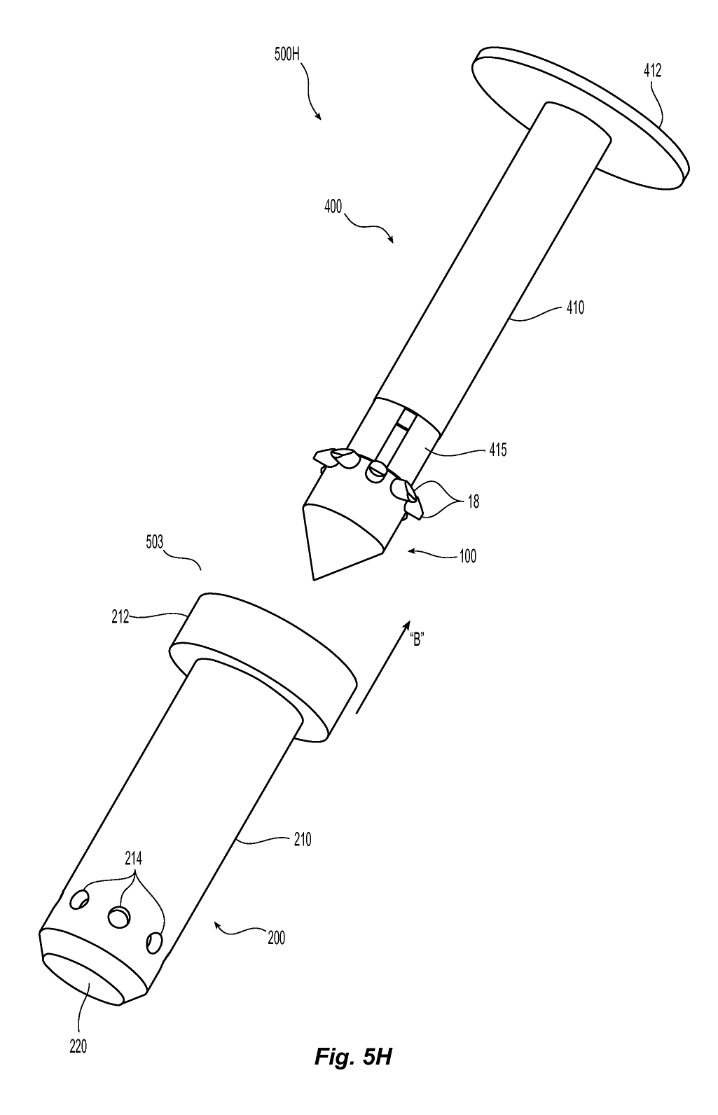

In FIGS. 1A-1C, the obturator 100 includes an obturator tip 12 at a distal end 13 of the obturator body 10. The obturator 100 further includes a plurality of leg structures 14 at a proximal end 11 of the obturator body 10. The leg structures 14 may be referred to as leg extensions. The plurality of leg structures 14 is configured to engage latching mechanisms 417 of a retractor 400 (discussed below with reference to FIGS. 4A-4D) via a plurality of snap profiles 16. The obturator 100 also includes a plurality of circumferential tabs 18 equally spaced apart from each other (or equidistant). Each tab 18 includes an angled or chamfered portion 19. The plurality of circumferential tabs 18 is configured to interlock with a plurality of openings 214 of a cannula 200 (discussed below with reference to FIGS. 2A-2C). The angled or chamfered portion 19 of each tab 18 facilitates removal of the tab 18 from the corresponding opening 214 as described below in connection with FIGS. 5D and 5H.

The plurality of circumferential tabs 18 may include eight separate and distinct tabs. However, in another exemplary embodiment the plurality of circumferential tabs 18 may include four separate and distinct tabs. One skilled in the art may contemplate any number of tabs 18 for connecting to a cannula 200 (discussed below). Additionally, the tabs 18 may be referred to as pins, projections, locking detents, or latches. Moreover, the plurality of circumferential tabs 18 may be positioned substantially adjacent a distal end of the plurality of leg structures 14.

FIGS. 2A-2C present various views of a cannula 200 illustrating a plurality of openings 214 at a distal end thereof, according to an embodiment of the present disclosure.

In FIGS. 2A-2C, cannula 200 includes a cannula head 212 at a proximal end 211 and a plurality of circumferential openings 214 at a distal end 213. The cannula head 212 is connected to a cannula body 210. The plurality of circumferential openings 214 is positioned at a distal end of the cannula body 210. Moreover, the distal end 213 of cannula 200 includes an opening 220 (see FIG. 2C).

The plurality of circumferential openings 214 may include eight openings. However, in another exemplary embodiment the plurality of circumferential openings 214 may include four openings. One skilled in the art may contemplate any number of openings 214 for connecting to the obturator 100 (discussed above). The number of openings 214 in cannula 200 may, but does not necessarily correspond to the number of tabs 18 on obturator 100.

In use or operation, the obturator 100 is configured to engage an inner surface of the cannula 200. The plurality of circumferential tabs 18 is configured to lockingly engage the plurality of circumferential openings 214 of the cannula 200. Thus, a secure connection is created between the obturator 100 and the cannula 200. In other words, the obturator 100 is configured to interlockingly engage the inner surface of the cannula 200 during insertion of the cannula 200 within an incision in tissue. Therefore, during an initial stage of insertion of a cannula into the body of a patient, the obturator 100 is connected to the cannula 200 and such components move together as one unit. Thus, the obturator 100 and the cannula 200 are pre-assembled to form a single unit, as described below with reference to FIGS. 5A-5D. Stated differently, the cannula 200 is pre-assembled to include an obturator 100 secured therein (e.g., at a distal end of the cannula 200).

Additionally, it is noted that the obturator 100 is much smaller than conventional obturator assemblies in order to securely attach or connect to the inner surface of the cannula 200. The length of the obturator 100 is less than the length of the cannula 200. For example, the obturator 100 may be less than half the length of the cannula 200. In most configurations, it is contemplated that the obturator 100 occupies a small portion of the distal end of the cannula 200. Therefore, manufacturing costs may be reduced because the obturator 100 does not include any exterior housing or sleeves. Instead, the cannula 200 is manufactured to include a penetrating tip, such as the obturator 100, pre-assembled thereon. Thus, the obturator 100 is part of the cannula 200 itself. Moreover, the obturator 100 may have a first diameter and the cannula 200 may have a second diameter, where the first diameter is smaller than the second diameter, such that obturator 100 snugly fits within the distal end of the cannula 200.

Moreover, regarding the cannula 200, a seal (not shown) or a seal structure or seal assembly may be positioned at the proximal end 211 in order to prevent insufflation gasses from escaping when, for example, the cannula 200 is inserted through tissue. One skilled in the art may contemplate a plurality of different seals capable of sealing the proximal end 211 of cannula 200.

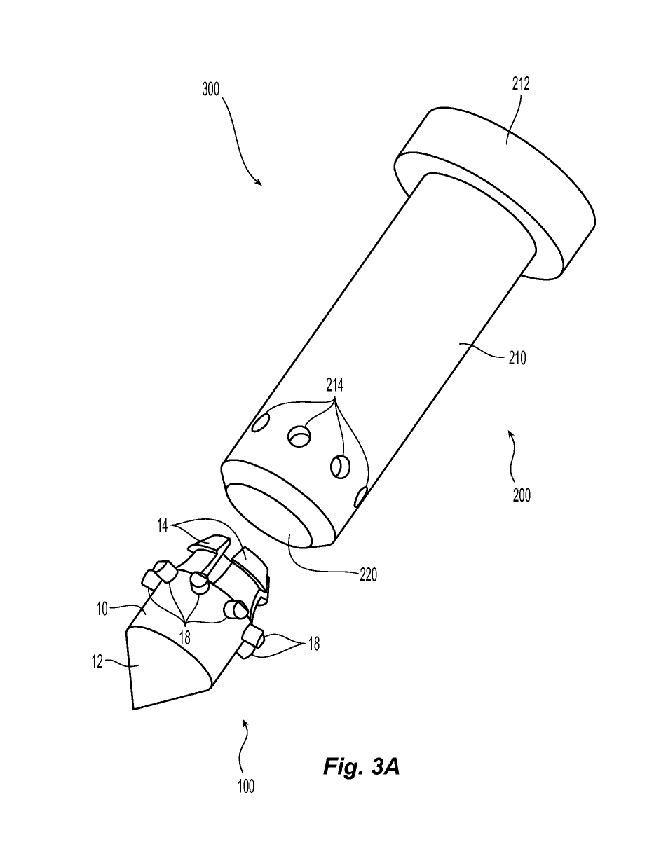

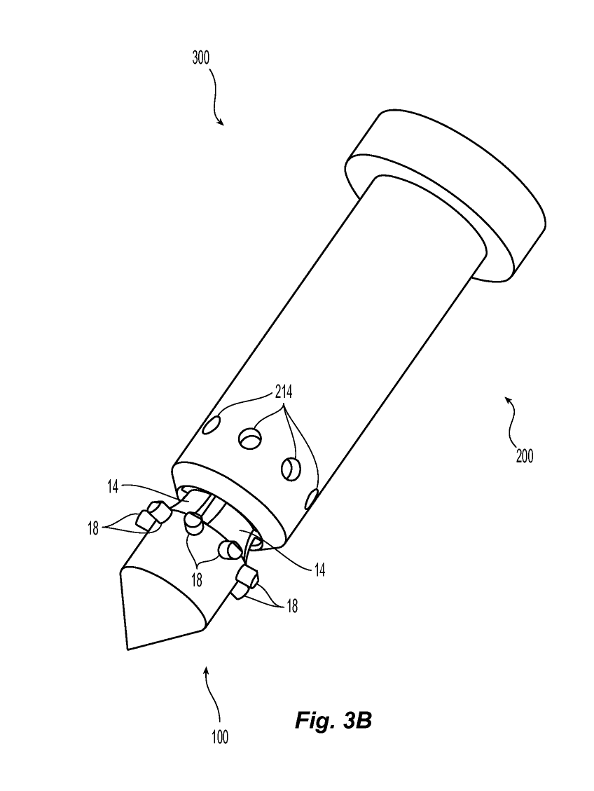

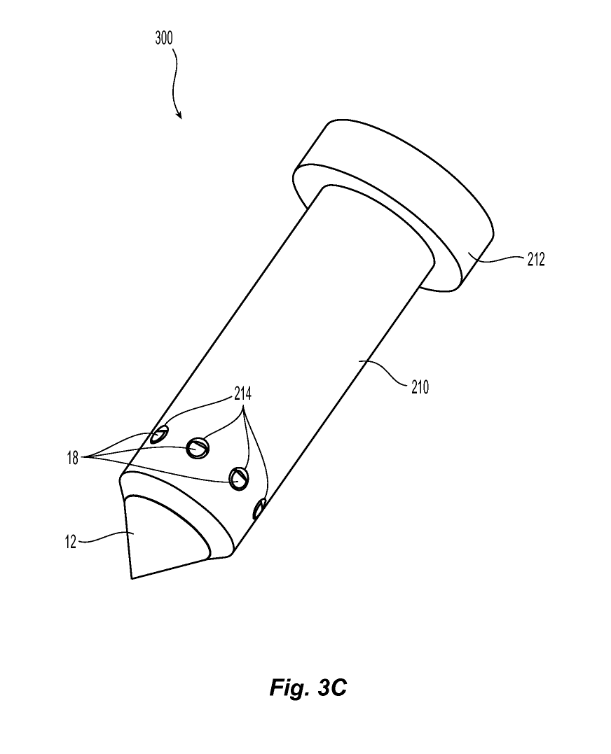

FIGS. 3A-3C present various views 300 of pre-assembling the obturator 100 of FIGS. 1A-1C to the cannula 200 of FIGS. 2A-2C, according to an embodiment of the present disclosure.

In FIG. 3A, the obturator 100 is shown separated from the cannula 200. In FIG. 3B, the obturator 100 is shown slowly inserted into the opening 220 of the cannula 200. The plurality of leg structures 14 is first inserted into the opening 220 of the cannula 200. In FIG. 3C, the obturator 100 is fully inserted into the distal end of the cannula 200. In the fully inserted position, each of the plurality of circumferential tabs 18 of the obturator 100 is inserted through one of the plurality of circumferential openings 214 of the cannula 200. Therefore, the obturator 100 securely interlocks with the cannula 200 at a distal end thereof. In the fully inserted position, only the tip 12 of the obturator 100 is exposed at the distal end of the cannula 200. The obturator body 10, as well as the plurality of leg structures 14 are fully inserted into the interior of the cannula 200.

FIGS. 4A-4D present various views of a retractor 400, according to an embodiment of the present disclosure.

In FIGS. 4A-4D, the retractor 400 includes a retractor body 410 having a retractor head 412 at a proximal end and a plurality of latching mechanisms 417 at a distal end. The back surface 415 of the plurality of latching mechanisms 417 extends the length of the retractor 400. The plurality of latching mechanisms 417 may be referred to as a plurality of collet fingers. The retractor 400 also includes openings 420 (see FIGS. 4B and 4C) at a distal end thereof, the openings 420 separating the plurality of latching mechanisms 417 from each other. The cross-sectional view of the retractor 400 illustrated in FIG. 4D, depicts the latching mechanisms 417 and the openings 420. Each of the plurality of latching mechanisms 417 includes an undercut portion 419 for connecting to the plurality of leg structures 14 of the obturator 100 (described below with reference to FIGS. 5A-5D).

FIGS. 5A-5H present various views 500A-500H of the retractor 400 entering the cannula 200, connecting to the obturator 100, and removing the obturator 100 from the cannula 200, according to an embodiment of the present disclosure.

In FIGS. 5A and 5E, in a first configuration 500A, the obturator 100 is pre-assembled to the cannula 200 at a distal end 501 of the cannula 200. The retractor 400 is shown positioned outside of the cannula 200 at a proximal end 503 of the cannula 200.

In FIGS. 5B and 5F, in a second configuration 500B, the retractor 400 is inserted at the proximal end 503 of the cannula 200. The refractor 400 moves in a direction “A” through channel 510 of cannula 200 to connect to the obturator 100. As shown, the body 410 of the retractor does not engage the interior surface 401 of the cannula 200.

It is noted that the plurality of leg extensions 14 is outwardly deflected with reference to axis “c-c,” whereas the plurality of latching mechanisms 417 is inwardly deflected with respect to axis “c-c.”

In FIGS. 5C and 5G, in a third configuration 500C, the distal end of the retractor 400 connects to the proximal end of the obturator 100. In particular, the plurality of leg structures 14 of the obturator 100 connect to the plurality of latching mechanisms 417 of the retractor 400. Stated differently, the snap profiles 16 of the plurality of leg structures 14 securely interconnect or interlock with the undercut portions 419 of the plurality of latching mechanisms 417 (see FIGS. 5A-5B). The undercut portions 419 sit directly below the snap profiles 16 to form the secure connection. In other words, the leg structures 14 are compressed or deflected when they come into contact with the latching mechanisms 417 during distal movement of the retractor 400. The latching mechanisms 417 inwardly deflect the leg structures 14 in order to enable a secure connection therebetween. In such an instance, the undercut portions 419 are directly adjacent the snap profiles 16. The undercut portions 419 may also be referred to as first surfaces and the snap profiles may be referred to as second surfaces, where the first and second surfaces engage each other to provide for the secure connection between the latching mechanisms 417 and the leg structures 14. The first and second surfaces are substantially parallel to each other when the secure connection between the latching mechanisms 417 and the leg structures 14 is enabled.

In FIGS. 5D and 5H, once the connection has been established between the retractor 400 and the obturator 100, the retractor 400 is moved in a direction “B,” in order to remove the obturator 100 from the cannula 200. In other words, the obturator 100 is temporarily or removably attached to the cannula 200. In use or operation, after the cannula 200 is inserted through an incision in tissue, the obturator 100 needs to be removed from the surgical site. The obturator 100 is removed to allow cannula 200 to act as an access port for surgical instruments. The retractor 400 is tasked with this responsibility. FIG. 5D illustrates how the obturator 100 is removed from the cannula 200. Upon movement of retractor 400 in direction “B”, the plurality of circumferential tabs 18 disengage from the plurality of circumferential openings 214, which disengagement is facilitated by the presence of angled or chamfered portion 19 on each tab 18. The plurality of circumferential tabs 18 may slidably engage the interior surface 401 of the cannula 200 during removal of the obturator 100 from the cannula 200.

Therefore, each of the plurality of leg structures 14 of the obturator 100 is configured to interlock with at least one latching mechanism 417 of the retractor 400 for disengaging the plurality of circumferential tabs 18 of the obturator 100 from the plurality of circumferential openings 214 of the cannula 200. Each of the plurality of leg structures 14 of the obturator 100 includes a snap profile 16 for engaging the at least one latching mechanism 417 of the retractor 400. Thus, in summary, the obturator 100 is pre-assembled to interlock the cannula 200, and the obturator 100, in its entirety, is positioned at a distal end of the cannula 200, such that the obturator 100 and the cannula 200 move together as a single unit. Stated differently, the obturator 100 is configured to be pre-assembled, in a releasable manner, into an inner portion 401 of the cannula 200. The retractor 400 is inserted into the cannula 200 to connect to the obturator 100, and then detach and fully remove the obturator 100 from the cannula 200.

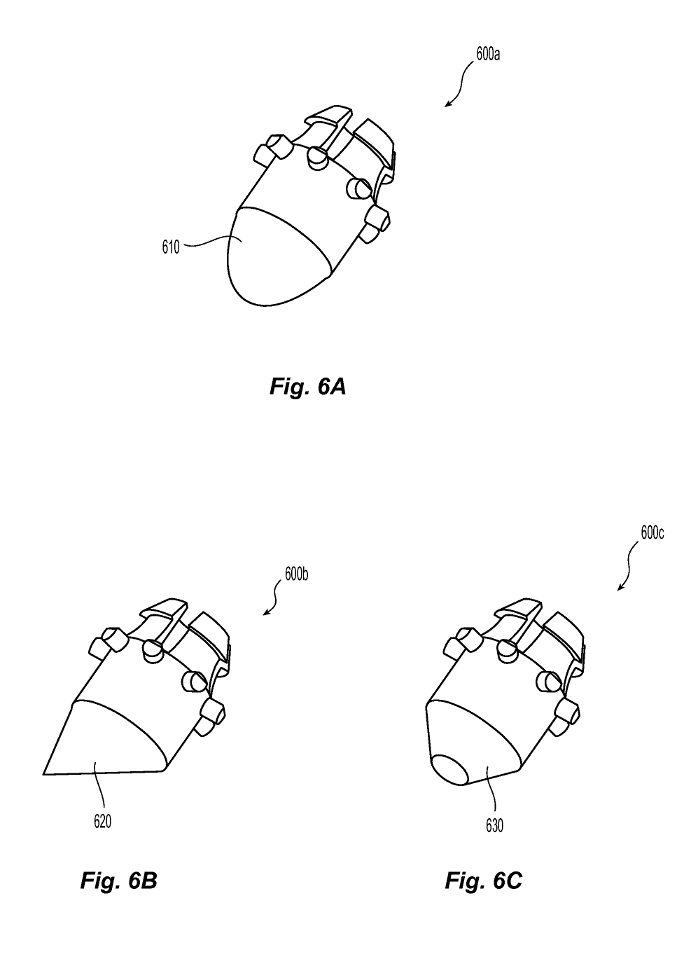

With reference to FIGS. 6A-6C, different obturators are presented, in accordance with embodiments of the present disclosure.

FIG. 6A illustrates an obturator 600a having a substantially circular tip 610.

FIG. 6B illustrates an obturator 600b having a off-center, substantially sharp tip 620.

FIG. 6C illustrates an obturator 600c having a truncated, substantially conical tip 630.

It is contemplated that any of these obturator tips 600a, 600b, 600c could be incorporated into the exemplary embodiments described with reference to FIGS. 1A-5H.

In summary, it is proposed to incorporate or embed a small obturator within or onto an interior surface of a cannula. The obturator includes a plurality of tabs that cooperate with a plurality of openings at a distal end of a cannula in order to provide a secure connection therebetween. The obturator and the cannula are considered a single unit and are constructed or formed in such a manner so as to move together or in tandem. Stated differently, the obturator and cannula form a single, unitary structure, where the two components are removably attached or connected to each other. The obturator may be removed from the cannula via, for example, a retractor including a plurality of finger collets that engagingly interlock with a plurality of leg extensions of the obturator to properly remove the obturator from the cannula.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of presently disclosed embodiments. Thus the scope of the embodiments should be determined by the appended claims and their legal equivalents, rather than by the examples given.

Persons skilled in the art will understand that the devices and methods specifically described herein and illustrated in the accompanying drawings are non-limiting exemplary embodiments. The features illustrated or described in connection with one exemplary embodiment may be combined with the features of other embodiments. Such modifications and variations are intended to be included within the scope of the present disclosure. As well, one skilled in the art will appreciate further features and advantages of the present disclosure based on the above-described embodiments. Accordingly, the present disclosure is not to be limited by what has been particularly shown and described, except as indicated by the appended claims.

It should be understood that the foregoing description is only illustrative of the present disclosure. Various alternatives and modifications can be devised by those skilled in the art without departing from the disclosure. Accordingly, the present disclosure is intended to embrace all such alternatives, modifications and variances. The embodiments described with reference to the attached drawing figs. are presented only to demonstrate certain examples of the disclosure. Other elements, steps, methods and techniques that are insubstantially different from those described above and/or in the appended claims are also intended to be within the scope of the disclosure.

What is claimed is:

1. A cannula assembly comprising:
a cannula having a plurality of circumferential openings at a distal end thereof; and
an obturator having a proximal end and a distal end, and a plurality of circumferential tabs that lockingly engage the plurality of circumferential openings of the cannula.

2. The cannula assembly according to claim 1, wherein the distal end of the obturator includes a tip and the proximal end of the obturator includes a plurality of leg structures.

3. The cannula assembly according to claim 2, wherein each of the plurality of leg structures of the obturator is configured to interlock with at least one latching mechanism of a retractor for disengaging the plurality of circumferential tabs of the obturator from the plurality of circumferential openings of the cannula.

4. The cannula assembly according to claim 2, wherein each of the plurality of leg structures of the obturator includes a snap profile for engaging the at least one latching mechanism of a retractor.

5. The cannula assembly according to claim 1, wherein the obturator is pre-assembled to interlock with the cannula.

6. The cannula assembly according to claim 1, wherein the obturator is positioned at the distal end of the cannula, such that the obturator and cannula move together as one unit.

7. The cannula assembly according to claim 1, wherein the cannula has a first length and the obturator has a second length, the second length being less than half of the first length.

8. An obturator, comprising:
a tip at a distal end thereof;
a plurality of leg extensions at a proximal end thereof; and
a plurality of circumferential tabs positioned between the proximal and distal ends of the obturator.

9. The obturator according to claim 8, wherein the plurality of circumferential tabs is adapted and dimensioned to engage a plurality of circumferential openings of a distal end of a cannula.

10. The obturator according to claim 9, wherein the plurality of leg extensions is adapted and dimensioned to engage a plurality of collet fingers of a retractor for removing the obturator from a cannula.

11. The obturator according to claim 10, wherein each of the plurality of leg extensions includes a snap profile for engaging the plurality of collet fingers of a refractor.

12. The obturator according to claim 8, wherein the obturator is configured to be releasably attached in an inner portion of a cannula.

13. A cannula comprising:
a head portion;
a body portion having a proximal end and a distal end;
a plurality of circumferential openings at the distal end of the body portion; and
a removable tip incorporated at the distal end of the body portion, the removable tip having a plurality of circumferential tabs for releasably interlocking with the plurality of circumferential openings.

14. The cannula according to claim 13, wherein the removable tip is pre-assembled to interlock within an inner portion at the distal end of the body portion.

15. The cannula according to claim 13, wherein the removable tip is an obturator.

16. The cannula according to claim 13, wherein the removable tip has a proximal end and a distal end, the distal end being a blunt tip and the proximal end being a plurality of leg extensions.

17. The cannula according to claim 16, wherein the plurality of leg extensions is adapted and dimensioned to engage a plurality of collet fingers of a refractor.

18. The cannula according to claim 13, wherein the body portion has a first length and the removable tip has a second length, the second length being less than one quarter of the first length.

19. A method comprising:
inserting a cannula assembly through a body wall of a patient, the cannula assembly including a cannula having a proximal end and a distal end and a plurality of circumferential openings at a distal end thereof and an obturator having a proximal end and a distal end, a plurality of circumferential tabs that engage the plurality of circumferential openings of the cannula and a plurality of leg extensions at a proximal end thereof, wherein the cannula has a first length and the obturator has a second length, the second length being less than half of the first length;
removing the obturator from the cannula by inserting a retractor into the proximal end of the cannula, the retractor including at least one latching mechanism that interlocks with the plurality of leg structures of the obturator and disengages the plurality of circumferential tabs of the obturator from the plurality of circumferential openings of the cannula; and
using the cannula as an access port for passage of surgical instruments through a body wall of a patient.
